(19) United States
(12) Patent Application Publication
(10) Pub. No.: US 2017/0290783 A1
(43) Pub. Date: Oct. 12, 2017

(54) Rabbit Maternal Pheromones to Enhance the Weight of Weaning Piglets

(71) Applicant: Animal Biotech, LLC, Dallas, TX (US)

(72) Inventor: John J. McGLONE, Lubbock, TX (US)

(21) Appl. No.: 15/092,684

(22) Filed: Apr. 7, 2016

(51) Int. Cl. A61K 31/11; G01G 17/08; A23K 50/30; A23K 20/105; A61K 9/00; A23K 50/60

(52) U.S. Cl. CPC A61K 31/11; A61K 9/0056; A23K 50/60; A23K 50/30; A23K 20/105; G01G 17/08

(57) ABSTRACT

There is provided an exemplary method of treating weaning piglets to induce weight gain, or to prevent a reduction in rate of weight gain, the method comprising exposing the weaning piglets to a rabbit maternal pheromone having an interomone effect. In an exemplary embodiment, the rabbit maternal pheromone is trans-2-methyl-2-butenal. The method may include the step of applying the rabbit maternal pheromone to a feeder for the weaning piglets. Moreover, the rabbit maternal pheromone may be applied to a feeder (or to the feed) for the weaning piglets at least once during a first week of weaning. Further, the method may include weighing the weaning piglets at intervals to determine a rate of weight gain, and measuring an amount of feed consumed by the weaning piglets to calculate the amount of weight gained per unit of feed consumed.

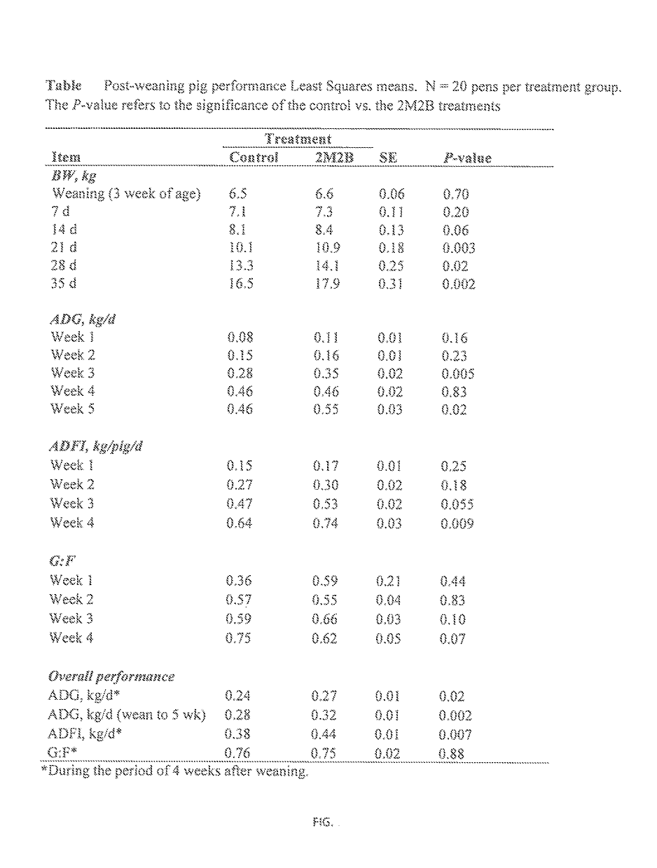

### BACKGROUND

### 1. Field of the Invention

The present invention relates to compositions and methods of increasing the weight gain of weaning piglets in terms of weight gain per unit of feed, and more particularly relates to exposing weaning piglets to rabbit maternal pheromones to cause an increase in weight gain per unit of feed.

### 2. Description of the Related Art

It has been found that the weaning stage of a piglet is particularly significant in that it interrupts a desired rate of growth (in weight) of the piglet. It has been speculated that weaning in itself imposes stress on the piglet, and this stress in turn results in a decreased rate of weight gain.

In modern pig farming, which is a large-scale controlled enterprise where costs and benefits are continually assessed, an interval of reduced weight gain during the weaning process is significant in adding to the costs of maintaining the piglets. During this weaning interval, despite that the piglets are fed and that their health is managed, weight gain is subpar per unit of feed intake. Because of this, the overall time period until they are within a size or weight range to be harvested, is extended. This also reduces the capacity of the farm by reducing the turnaround time of the farm. Capacity increases then require an extension of buildings and related equipment, which adds capital costs. If the turnaround time (i.e. the time for the piglets to reach harvest size) is reduced, more piglet-raising cycles can be obtained within the same farm, without the need for additional capital investment. Thus, the weaning interval of lowered weight gain has cost implications as to both direct costs (feed) as well as capital.

There is a significant financial incentive to improve weight gain per unit of feed during the weaning period to effectively reduce time to achieve the harvest weight. But thus far efforts have not yielded results that may be regarded as uniformly successful and that provide a positive cost-benefit analysis.

### SUMMARY

In an exemplary embodiment, there is provided a method of treating weaning piglets to induce weight gain, or to prevent a reduction in rate of weight gain, the method comprising exposing the weaning piglets to a rabbit maternal pheromone having an interomone effect. In a further exemplary embodiment, the rabbit maternal pheromone is trans-2-methyl-2-butenal. The method may include the step of applying the rabbit maternal pheromone to a feeder for the weaning piglets. Moreover, the step of exposing to the rabbit maternal pheromone may include applying the rabbit maternal pheromone to a feeder for the weaning piglets at least once during a first week of weaning. Further, the method may include weighing the weaning piglets at intervals to determine a rate of weight gain, and measuring an amount of feed consumed by the weaning piglets to calculate the amount of weight gained per unit of feed consumed.

In another exemplary embodiment, there is provided a method of introducing into an environment of weaning piglets a formulation comprising a rabbit maternal pheromone having interomone properties, the formulation effective to improve a rate of weight gain per unit of feed in weaning piglets. The formulation may be in liquid, in feed, or in pelletized form. When in liquid form, the formulation may include an alcohol, such as propanol. In pelletized form, the formulation may be of pellets treated with the liquid formulation. In liquid form, the concentration of the rabbit maternal pheromone in the formulation may be about 1.0 μg/L, albeit that the concentration or the amount of pheromones is not a limiting factor. The pheromone should be present in an amount detectable to the olfactory senses of the weaning piglets. The formulation may include the interomone trans-2-methyl-2-butenal. The formulation may be effective upon a single exposure of weaning piglets to the formulation.

The foregoing is a brief summary of some aspects of the technology presented herein, and does not limit the scope of the inventions, which are described in the claims here below.

### BRIEF DESCRIPTION OF THE DRAWING

FIG. is a Table depicting results of the Example in tabular layout.

### DETAILED DESCRIPTION

The following description relates to the use of formulations of rabbit maternal pheromones that are useful in improving the weight gain (or put another way, reducing the weight loss) of piglets undergoing weaning. Piglets are commonly weaned about 21 days of age. While not bound by any theory, weaning is often theorized to cause stress in piglets due to the attendant changes in diet and physical and thermal environments. Importantly, weaned piglets also experience a change of social and olfactory environments that contribute to the stress of weaning. Weaning is well-known to cause weight loss, increased blood cortisol concentrations, a depressed immune system and significant behavioral changes.

Antibiotics are commonly used in weaned pigs to reduce the negative effects of weaning. However, social concerns about the use of antibiotics have led to a search for natural (in the sense of non-synthetic that occur in the natural environment) alternatives.

Pheromones might be useful to modify behavior of pigs because pigs rely so heavily on olfactory signals. The boar pheromone (Androstenone) reduced pig aggressive behaviors and can temporarily improve weight gain of re-grouped, stressed pigs. A putative maternal pheromone that is a mixture of fatty acids found on the mammary surfaces of lactating sows also was shown to increase post-weaning weight gain.

Another class of semiochemicals is the interomones. These compounds are a pheromone in one species, but have a different effect on the behavior or physiology of another species. The inventors have previously considered how interomones may be utilized beneficially.

Upon conducting experiments on the possible effects of the rabbit maternal pheromone (2-methyl-2-butenal; also known as “2M2B,” or tiglic aldehyde) in the post-weaning environment on weaned pig performance and behavior, a surprising and unexpected discovery was made. It was found, as detailed below, that the exposure of the weaning piglets to rabbit maternal pheromones countered the expected weight loss of the piglets during the weaning period and led to an increase of weight relative to control weaning piglets that were not exposed to the pheromones.

When in liquid form, the formulation may include an alcohol, such as propanol, as a carrier. In pelletized form, the formulation may be of pellets treated with the liquid formulation. In liquid form, the concentration of the rabbit maternal pheromone in the formulation may be about 1.0 μg/L, albeit that the concentration or the amount of pheromones is not a limiting factor. The rabbit maternal pheromone may also be in the feed or water supplied to the weaning piglets. The pheromone should be present in an amount detectable to the olfactory senses of the weaning piglets.

It is speculated that the piglet post-weaning environment is deficient in familiar and perhaps comforting maternal odors. While not bound, it is hypothesized that part of the post-weaning lag in growth and challenges in piglet health is due to a sudden loss of maternal odors that are meaningful to the piglet.

Trans-2-methyl-2-butenal is naturally found in berries, some vegetables, chicken fat and of course in mammary secretions of rabbits. It is sold as a food-grade flavoring agent. Trans-2-methyl-2-butenal is listed as generally recognized as safe by the flavor and extract manufacturers association of the USA (FEMA, 2016).

In the rabbit, trans-2-methyl-2-butenal induces nipple search behavior in rabbit pups. We have examined pheromones and interomones that might provide a maternal odor in the post-weaning environment. To be called a pheromone, trans-2-methyl-2-butenal would have be found to be in one pig (perhaps the mother), and to have an effect on the physiology or behavior of another pig. None of the criteria for a definition of a pheromone have been identified in the pig. While that may happen over time, for now, we speculate that trans-2-methyl-2-butenal is acting as an interomone in the pig.

An interomone is a molecule, or collection of molecules, that may act as a pheromone in one species, but have a different effect in other species. For example, the pig sex pheromone that stops dogs from barking is an interomone.

Semiochemicals are conserved across diverse species. For example, the bark beetle aggregation pheromone is a sex pheromone in elephants. It is speculated that different species cannot easily use the same pheromone for the same purpose in the same ecosystem or there would be biological confusion. However, the same molecule may be used for different species for different purposes without invoking confusion. Secondly, volatile compounds are conserved over plant and animal species (like trans-2-methyl-2-butenal), and a particular molecule may serve different functions.

As far as we are aware, the rabbit maternal-neonatal molecule trans-2-methyl-2-butenal has not been shown to be a pheromone in the pig. It has to be viewed as an interomone then in the effect that it has on weaning piglets.

Interomones, like trans-2-methyl-2-butenal, are natural, powerful at low concentrations, and generally safe, in part because they are natural and in part because they are sprayed on the environment and not injected. All that is required is a sufficient amount of the rabbit maternal pheromone in the environment for the weaning piglets to be able to sense the pheromone with olfactory senses. Such a concentration and amount may be quite low.

The interomone trans-2-methyl-2-butenal might serve as an alternative to administering medically-important antibiotics as growth-promoting agents. This interomone does not act as a drug, but is a new class of natural compounds when seen in the context of weaning piglets. The inventor's theory that the post-weaning environment lacks maternal odors, which contribute to weaning stress, and that exposure to the rabbit maternal pheromone can reduce the negative effects associated with a lack of maternal odors, has surprisingly proven to be correct.

Trans-2-methyl-2-butenal, when added in a single application to the feeder in the post-weaning environment, was found to stimulate ADFI and ADG in piglets. The mechanism by which trans-2-methyl-2-butenal stimulates pig performance is not known definitively at this time. It is theorized, without being bound, that trans-2-methyl-2-butenal stimulates feeding behavior, feed intake and increased growth. The stimulatory effect on feed intake is very small (and not significant) during the first week after weaning, but it increases in magnitude in weeks two to five after weaning. During the period of increased growth, the trans-2-methyl-2-butenal that was applied is not likely to still be present, since it is volatile. It is therefore concluded that a single exposure to the rabbit maternal pheromone is all that is necessary for the positive observed effects.

The following example reflects an experiment carried out to test the initial hypothesis that rabbit maternal pheromones may have an effect as interomones on weaning piglets to either minimize weight loss or increase weight during the weaning period per unit of feed. The example is non-limiting of the inventions, and is presented to demonstrate the effectiveness of the inventions claimed here below.

### Example

Piglets (n=120) were randomly selected from nursing sows. A total of 120 piglets, 60 males and 60 females, were weaned at approximately 3 weeks of age and transported for four hours before entering the nursery site. Piglets were housed in groups of three, single-sex (either barrows or gilts), and had ad libitum access to feed and water. A total of 40 pens (the experimental unit) were available; 10 each of male and female experiencing control or pheromone therapy. Space allowance was in excess of industry standards (0.6 m² per piglet). Body weight and feed intake were each measured once per week from commencement of weaning until 35 days after weaning.

Control and pheromone-treated piglets were housed in separate rooms, with no airflow between the rooms to ensure no cross contamination between air spaces. To expose piglets to the rabbit maternal pheromone, test piglet feeders received 25 mL of the trans-2-methyl-2-butenal solution (1 μg/mL in isopropyl alcohol) while the control piglet feeders received 25 mL of isopropyl alcohol only. Feeders were sprayed immediately before the arrival of piglets to the nursery site.

Half of the pigs and pens in each group were transported and half were moved directly from the farrowing crates to the adjacent nursery building. The experimental design was a completely random design with a two by two factorial arrangement of transport (or not) and trans-2-methyl-2-butenal or placebo control.

Average daily gain (ADG, kg. per day) was calculated from the body weights both weekly and over the entire period. Average daily feed intake (ADFI, kg. per pig per day) was calculated based on weekly feed consumption. Gain to Feed (G:F) was calculated each week and over the 28-day post-weaning period.

### Results

Data were analyzed using SAS software (SAS, 2012). PROC Univariate was used to establish that the overall ADG, ADFI and G:F ratio data were normally distributed. The general linear models procedure was used with pen as the experimental unit. There were 40 experimental units (pens) in total. The original model contained the factorial effects of (A) trans-2-methyl-2-butenal applied during transport or not, (B) trans-2-methyl-2-butenal applied to the nursery feeder or not, and (3) pig sex (barrow vs. gilt) and all possible interactions. For overall performance measures, the effects of transport application of trans-2-methyl-2-butenal, sex and the interaction of these variables with trans-2-methyl-2-butenal applied in the nursery were not significant (P>0.10). A final simplified analysis was performed with only the effects of trans-2-methyl-2-butenal applied in the nursery or not with 20 pens per treatment.

For all performance measures, the interaction between transport and trans-2-methyl-2-butenal application was found not to be significant (P>0.10). Transport for 4 hours at the time of weaning did not have any long-term effect on pig performance. Sex effects and the sex by treatment interactions were also not significant, except for body weight data in week 1. In other weeks, and over the entire test period, the sex effects and sex effect interactions with other treatments were not significant.

The Table of the FIG. shows the results of pig performance measures. Feed intake data were not available for the period between 4 and 5 weeks after weaning, however body weights at 35 days after weaning were collected.

Piglet body weights were statistically similar at weaning and 7 days later. At 14 days after weaning, pigs experiencing trans-2-methyl-2-butenal treatment tended (P=0.06) to be heavier. On days 21, 28 and 35 after weaning, piglets exposed to trans-2-methyl-2-butenal were significantly heavier (P<0.05) than control pigs. Exposure to trans-2-methyl-2-butenal caused an increase (P<0.05) in overall ADG (during both the 4 and 5 week period after weaning) and ADFI during 4 weeks after weaning. The G:F was not influenced (P>0.10) by treatments. Exposure to trans-2-methyl-2-butenal increased post-weaning ADG in weeks 3, 5 and overall (P<0.05). ADFI was higher (P<0.05) among trans-2-methyl-2-butenal-treated pens in weeks 3 and 4 after weaning, and during the overall 5-week growth period. Gain to feed ratio was not significantly changed by trans-2-methyl-2-butenal. The effect of trans-2-methyl-2-butenal was that it stimulated feed intake and body weight gain. At weaning, control pigs weighed 6.5±0.06 kg and pig to experience trans-2-methyl-2-butenal weighed 6.6±0.025 kg (P>0.10). Five weeks later, control pigs weighed 16.5±0.31 kg while pigs experiencing trans-2-methyl-2-butenal weighed 17.9±0.31, an 8.5% improvement (P=0.01).

While examples of embodiments of the technology have been presented and described in text and some examples also by way of illustration, it will be appreciated that various changes and modifications may be made in the described technology without departing from the scope of the inventions, which are set forth in and only limited by the scope of the appended patent claims, as properly interpreted and construed.

1. A method of treating weaning piglets to induce weight gain, the method comprising exposing the weaning piglets to a rabbit maternal pheromone having an interomone effect, wherein the rabbit maternal pheromone is trans-2-methyl-2-butenal.

2. (canceled)

3. The method of claim 1, wherein the step of exposing includes applying the rabbit maternal pheromone to a feeder for the weaning piglets.

4. The method of claim 1, wherein the step of exposing includes applying the rabbit maternal pheromone to a feeder for the weaning piglets at least once during a first week of weaning.

5. The method of claim 1, further comprising weighing the weaning piglets at intervals to determine a rate of weight gain.

6. The method of claim 5, further comprising measuring an amount of feed consumed by the weaning piglets to calculate the amount of weight gained per unit of feed consumed.

7. A method of treating weaning piglets to induce weight gain, the method comprising exposing the weaning piglets at least once in the first week of weaning to a rabbit maternal pheromone comprising trans-2-methyl-2-butenal.

8. The method of claim 7, further comprising weighing the weaning piglets at intervals to determine a rate of weight gain.

9. The method of claim 8, further comprising measuring an amount of feed consumed by the weaning piglets to calculate the amount of weight gained per unit of feed consumed.

10. A method of treating an environment of weaning piglets, the method comprising introducing into the environment a rabbit maternal pheromone having interomone properties, the rabbit maternal pheromone effective to improve a rate of weight gain per unit of feed in weaning piglets, wherein the rabbit maternal pheromone is trans-2-methyl-2-butenal.

11. (canceled)

12. The method of claim 10, wherein the rabbit maternal pheromone is effective upon exposure of weaning piglets to the rabbit maternal pheromone in the environment.

13.

14. The method of claim 10, wherein the rabbit maternal pheromone is effective upon a single exposure of weaning piglets to the rabbit maternal pheromone in the environment.

15. The method of claim 10, wherein the rabbit maternal pheromone is in a liquid formulation.

16. The method of claim 15, wherein the formulation has about 1.0 μg/L of trans-2-methyl-2-butenal.

17. The method of claim 10, wherein the formulation includes an alcohol.

18. The method of claim 17, wherein the formulation has about 1.0 μg/L of trans-2-methyl-2-butenal.

19. The method of claim 10 wherein a concentration of the rabbit maternal pheromone in the environment is detectable to the olfactory senses of the weaning piglets.

20. The method of claim 10, wherein the rabbit maternal pheromone is in a feed formulation.

21. The method of claim 7, wherein the rabbit maternal pheromone is in a liquid formulation.
